(19) United States
(12) Patent Application Publication
(10) Pub. No.: US 2023/0017920 A1
(43) Pub. Date: Jan. 19, 2023

(54) SHELF-STABLE HYPOCHLOROUS ACID

(71) Applicant: Emmett Manuel CUNNINGHAM, Glen Allen, VA (US)

(72) Inventor: Emmett Manuel CUNNINGHAM, Glen Allen, VA (US)

(21) Appl. No.: 17/757,791

(22) PCT Filed: Dec. 26, 2020
(86) PCT No.: PCT/US2020/067062

§ 371 Date: Jun. 21, 2022

Related U.S. Application Data
(60) Provisional application No. 62/974,810, filed on Dec. 26, 2019.

(51) Int. Cl. A61K 33/20; C01B 11/04; A61K 9/00

(52) U.S. Cl. CPC A61K 33/20; C01B 11/04; A61K 9/0073; A61K 9/0043

(57) ABSTRACT

The use of powdered or gelled hypochlorous acid (HOCI) in the treatment or prevention of viral and bacterial infection is disclosed.

### CROSS-REFERENCE TO RELATED APPLICATIONS

This application claims priority to U.S. Provisional Patent Application Ser. No. 62/974,810, filed 26 Dec. 2019, which is hereby incorporated herein as though fully set forth.

### TECHNICAL FIELD

The present invention is related to the treatment or prevention of infection and, more particularly, to the treatment or prevention of viral or bacterial infection diseases by chlorination using liquid or gelled hypochlorous acid in the form of a nasal spray or nasal irrigation solution or as a liquid or gel wound spray or dressing.

### BACKGROUND

Viral infections in humans include the common cold, primarily caused by rhinoviruses, picornaviruses, and coronaviruses, although over 200 different viral types are known to cause colds. Because there are more than 200 viruses that cause the common cold, the human body can never build up resistance to all of them. This is why colds are so common and often return. According to the CDC (Centers for Disease Control and Prevention), adults get 2-3 colds per year, and children may have up to 12 per year.

Other viral infections include influenza and other infectious diseases with similar symptoms. Influenza viruses known to infect humans include three genera: influenza virus A (including the species H1N1, H2N2, H3N2, HSN1, H7N7, H1N2, N9N2, H7N2, H7N3, and H10N7), influenza B (consisting of a single species), and influenza C (consisting of a single species). Collectively, colds, influenza, and infectious diseases with similar symptoms are referred to as influenza-like illnesses.

Often, viral infection begins with deposition of virus/bacteria into the front of the nasal passages. This can occur via contaminated fingers, inhalation of droplets containing the virus, or other mechanisms. Such deposition by as few as one colony forming unit (CFU) is sufficient to produce an infection in the individual.

From the front of the nasal passages, the virus/bacteria are transported to the back of the nose and onto the turbinate shelves and adenoid area, often by the mere passage of air during inhalation. The adenoid is a lymph gland structure that contains cells to which cold viruses attach. The virus/bacteria then attach to surface receptors (e.g., Inter-Cellular Adhesion Molecule 1 (ICAM-1) in the case of the virus causing the common cold) on individual cells, enter the cells, replicate, and rupture the cells, spreading the virus to other cells and repeating the infection cycle. Replication of a virus causing the common cold typically begins eight to 12 hours after infection, with symptoms beginning between 10 hours and five days post-infection and peaking two to three days after onset.

While infection by cold virus typically results in much less severe consequences in terms of an individual' s health, the overall economic cost of the common cold, including the costs of treatment and lost productivity, is estimated to be over $20 billion per year in the United States alone. In addition, the widespread but futile use of antibiotics to treat cold symptoms has greatly increased antibiotic resistance, the consequences of which, in terms of both dollars and lives, are difficult or impossible to accurately estimate.

Known methods of treating symptoms of cold and influenza, such as the use of saline nasal sprays, dry nasal passages and often lead to irritation. In addition, the use of saline sprays alone is believed to provide a more hospitable environment for viral replication.

In addition to the treatment or prevention of viral/bacterial infection diseases in the nasal passages, liquid or gelled compositions containing hypochlorous acid (HOCl) can and should be used to facilitate the healing of wounds, both acute and chronic. The time period during which such wounds have been in existence and their tendency to heal properly can relate directly to the viral or bacterial infections on the surface of the wound. The role of colonization in delaying wound healing is uncertain but is most commonly ascribed to aerobic or facultative species, such as Staphylococcus aureus, Pseudomonas aeruginosa, and beta-hemolytic streptococci. The reported role of anaerobic species could be related more to the omission of anaerobic culture strategies in studies than to lack of virulence. There are, also, many types of skin ulcers which can lead to infected ulcers, once open to the environment. In order to stem the use of antibiotics, which can lead to untreatable staph infections (staph is one of the most common causes of skin infections in the U.S.), other effective treatments are essential. Critical colonization is also referred to as increased bacterial burden or covert infection. Substantial colonization might or might not cause the obvious signs of inflammation but will likely affect wound healing, with failure to heal or slowing of progression. The elimination of virus/bacteria forming colonies by using liquid or gelled compositions containing HOCI provide an effective way to treat or prevent viral or bacterial infection.

Most chronic wounds are related to diabetes mellitus, venous stasis, peripheral vascular diseases, and pressure ulcerations. An open wound is a favorable niche for bacterial colonization and infection. Infection in chronic wounds starts with contamination, then colonization and critical colonization take place before an infection forms.

Biofilm formation is now recognized as a serious problem in chronic wound infections. A biofilm is a complex structure of microorganisms that generate a protective shell, allowing bacteria to collect and proliferate. Most of the microorganisms that form biofilms can also be found growing in microbial infections. The same species of bacteria have significant differences in existence that range from free floating to living within the biofilm. The biofilm structure of microorganisms renders phagocytosis difficult, increases resistance to antibiotics, and adheres to unfavorable niches such as chronic wounds.

One of the remarkable features of the immune system against invading pathogens is its ability to generate an effective and rapid response by developing a group of highly reactive chemicals, such as reactive oxygen species (ROS). The mitochondrial membrane bound enzyme nicotinamide adenine dinucleotide phosphate-oxidase (NADPH) is a primary enzyme responsible for ROS production. During the activation of neutrophils, respiratory bursts generate hydrogen peroxide (H₂O₂) and the activated granule enzyme myeloperoxidase converts H₂O₂ to HOCl in the presence of Cl⁻ and H⁺. hypochlorous acid leads to cell death by the oxidation of sulfhydryl enzymes and amino acids, ring chlorination of amino acids, loss of intracellular contents, decreased uptake of nutrients, and inhibition of protein synthesis, decreased oxygen uptake, oxidation of respiratory components, decreased adenosine triphosphate production, breaks in DNA, and depressed DNA synthesis.

hypochlorous acid is highly active against all bacterial, viral, and fungal human pathogens and a small amount of HOCl can kill spore-forming and non-spore bacteria in a short time period.

Since most of the etiologic factors in chronic wound infections are forming biofilm, and most of the topical antiseptics impair wound healing with their cytotoxic effect, therapeutic strategies against biofilms with high microbial eradication and good wound healing effects will decrease the morbidity and mortality rates of patients and reduce the attendant economic burden.

According to embodiments of this invention, a hypochlorous acid liquid or gel pack (HLGP) can penetrate and remove biofilms and end the reproduction cycles of all the microorganisms that habitat it and under it, within seconds.

Testing supports stabilized HOCl solutions as a preferred wound care and nasal infection solution with powerful and rapid killing effects on different types of microorganisms, biofilms, and antimicrobial biocide effects within the biofilm.

This reduces the use of antibiotics that lead to more “super germs” like Methicillin-resistant Staphylococcus aureus (MRSA).

By using a hypochlorous acid composition to treat wounds, especially ulcers, as soon as possible, one would avoid the possibility of the bacteria going deeper into the body or bloodstream, resulting in a need for antibiotic treatment, while also ensuring effective treatment of the wound surface.

### SUMMARY

The invention provides methods and systems for treating or preventing viral/bacterial infectious diseases.

In one embodiment, the invention provides a method of treating or preventing viral infection in an individual, the method comprising: administering to the individual, via at least one nasal cavity, liquid hypochlorous acid.

In another embodiment, the invention provides a method of treating or preventing bacterial infection in an individual, the method comprising: administering to the individual's wounds or ulcers a quantity of liquid hypochlorous acid.

In still another embodiment, the invention provides a powdered composition comprising: one or more of: lithium hypochlorite, calcium hypochlorite, or sodium hypochlorite; sodium chloride; and an acid.

The illustrative aspects of the present invention are designed to solve the problems herein described and other problems not discussed which are discoverable by one skilled in the trade.

### DETAILED DESCRIPTION

The invention provides methods and systems for treating or preventing viral/bacterial infection by chlorination using a liquid or gelled HOCl composition. In some embodiments, the liquid or gelled HOCl composition may be nebulized or added to a dressing. In other embodiments, the liquid or gelled HOC1 composition may be used for nasal irrigation.

As used herein, the terms “treating,” “treat,” “treatment,” “prevent,” “prevention,” and “preventing” refer to an ameliorative or prophylactic effect on viral/bacteria count, viral/bacterial infection, or a symptom of viral/bacterial infection.

A liquid or gelled HOC1 composition suitable for use in practicing various embodiments of the invention includes a shelf-stable HOCl manufactured from a powdered mixture or combination of lithium hypochlorite, calcium hypochlorite, and/or sodium hypochlorite; an acid such as ascorbic acid or citric acid; and sodium chloride, with the addition of an inorganic emulsifier if the gelled form is being prepared. The resulting HOCl has a pH approximately the same as or near the pH of the nasal cavity or and open wound, which is about 5-6.5.

Such an HOCl solution will have an indefinite shelf life, as it is prepared and stored as a powder and only mixed with a quantity of water when needed and used.

sodium chloride's effect on the action of HOCl composition may simply be the chloride portion being made available to replenish chloride molecules of the HOCl as they are consumed by contact with the organic material on the surface of the affected area. At a pH of 5-6.5, the HOCl composition is right at 100% of the chloride molecules and would need replenishing as these are used up by the organic material on contact. sodium chloride also has a drying effect on an open wound or ulcer.

When table salt (NaCl) is dissolved in water, something interesting happens. Because water is polar, it has electrostatic interactions with NaCl. Enough of the solvent water molecules are able to gather around the salt such that the combined electronegativity of water is enough to rip the sodium from the chloride ion. Na⁺ is surrounded by the oxygen (negative) end of the water molecule, and Cl⁻ is surrounded by the hydrogen (positive) ends of the water molecule. In this sense, water is physically separating the salt atoms from each other. This process leaves the Cl³¹ molecules available to the acidic levels of the solution (5-6.5 pH) creating more HOCl as needed.

The active ingredient in compositions according to the invention is HOCl, a molecule already present in the human body and used by white blood cells to attack microorganisms. White blood cells (specifically neutrophils) respond to tissue invasion by migrating to the site of an infection. Neutrophils seek out pathogens such as bacteria or viruses, surround them, and destroy them using HOCl. This process is known as phagocytosis. Phagocytosis is involved in the acquisition of nutrients for some cells, and in the immune system it is a major mechanism used to remove pathogens and cell debris. Bacteria, dead tissue cells, and small mineral particles are all examples of objects that may be phagocytosed. Likewise, sodium chloride is also present in the human body and has no side effects.

Furthermore, HOCl attacks the metabolic functions of microorganisms, disrupting their DNA structure, rendering them unable to build any resistance to HOCl. Various studies (H Park, et al. “Effects of Chlorine and pH on Efficacy of Electrolyzed Water for Inactivating Escherichia coli O157:H7 and Listeria monocytogenes,” International Journal of Food Microbiology, 91 (2004), 13-18) have shown that HOCl kills individual bacterial cells within seconds.

HOCl is used for:
Acute and chronic dermal lesions;
Stage I—IV pressure ulcers;
Stasis ulcers—diabetic ulcers;
First- and second-degree burns; and
Abrasions and minor irritations of the skin

HOCl is also very effective in the reduction of odors associated with bacteria.

The hypochlorite ion carries a negative electrical charge, while HOCl carries no electrical charge. hypochlorous acid moves quickly, able to oxidize the bacteria in a matter of seconds, while the hypochlorite ion might take up to a half hour to do the same. Germ surfaces carry a negative electrical charge, which results in a repulsion of the negatively charged hypochlorite ion from the area of the germ surfaces, making the hypochlorite ion less effective at killing germs. The ratio of the two compounds is determined by the relative acidity (pH) of the water. HOCl compositions according to the invention automaticall adjusts the pH level (5-6.5) to make HOC1 more dominant, as it is more efficient at killing bacteria. Between a pH of 5 and 5.5, the HOCL is 100%. hypochlorous acid's lack of electrical charge allows it to more efficiently penetrate the protective barriers surrounding all microorganisms.

One advantage of the invention is the purity of all of its ingredients. Years of testing demonstrate that adherence to strict compliance of process and ingredients were the only solution, providing effective results through a dynamic equilibrium of all ingredients. Batch processing of medical and pharmaceutical grade lithium hypochlorite, calcium hypochlorite and/or sodium hypochlorite, an acid like ascorbic acid or citric acid, sodium chloride, and optionally an inorganic emulsifier provides a level of control to meet exacting standards in the product. These standards include guaranteed levels of free chlorine, purity, and repeatability. Introducing a chemical solution to a human/animal surface or cavity requires the solution to be accurate each and every time.

In some embodiments of the invention, the HOCl composition contains between about 10 ppm and about 500 ppm HOCl.

In some embodiments of the invention, the HOCl composition contains between about 10 ppm and about 1000 ppm of NaCl.

In some embodiments, the HOCl composition has a pH between about 2 and about 9.

In some embodiments, the HOCl composition has a pH between about 5 and about 6.5.

It is extremely important to treat the nasal passage with products that are similar in pH to those of the nasal cavity, as local pH may have a direct effect on the rate and extent of absorption of ionizable compounds. The HOCl composition of the invention includes positive sodium ions (Na⁺) and negative chloride ions (Cl⁻), making it an ionic compound and hence totally absorbable when it comes in contact with a surface at pH 5-6.5. The nasal cavity is slightly acidic and has a pH of 5-6.5.

In another embodiment of the invention, the HOCl composition is used in a light gelled form. That is, the composition may be used in a free-flowing light viscous form. A technique for increasing the viscosity and the surface tension of a liquid is adding 0.5-5.0% of a FDA approved non-organic emulsifier to a liquid HOCl composition that will not disrupt its stability. Water-soluble emulsifiers have a good rate of surface tension, are very silky and will increase the wet surface contact life of the HOCl composition by its nature of the high surface tension which allows the air flow to pass over it and stay wetter longer with good skin contact.

In some embodiments of the invention, the HOCl composition is used in a liquid or gelled form. That is, the HOCl composition may be used in a free-flowing form or in a slight viscous form. Any known or later-developed technique for increasing the viscosity of a liquid, FDA approved for human consumption and not reducing its effectiveness, may be employed to produce a gelled HOCl composition suitable for use in practicing embodiments of the invention.

Contact with the liquid or gelled HOCl composition has been shown to kill various viruses and bacteria within seconds. As such, administration of the liquid or gelled

HOCl composition to the nasal cavities will kill virus particles within the nasal cavities before their infection of cells reach the adenoid area. In addition, inhalation or drainage will carry a portion of the liquid or gelled HOCl composition deeper into the nasal cavity and on to the adenoid area, where the antiviral and antibacterial activity of the HOCI will kill virus/bacterial particles that may have already reached the adenoid area.

Viral infection may be treated or prevented, according to some embodiments of the invention, by administering the liquid or gelled HOCl composition to an individual via a nasal cavity. For example, the liquid or gelled HOCl composition may be applied to a swab or contained within a syringe, which is then used to introduce the HOCl composition to the nasal cavity. This is best done in the prone position, or head held far back, in order to fully coat the adenoid area and the turbinate shelves.

In other embodiments it can be administered by means of nasal irrigation through the use of a syringe, neti pot, nose cleaner with powered suction neti or a squeeze bottle. The HOCl composition will run through a patient's nasal passages over the turbinate shelves and onto the adenoid area and drain out of the mouth or other nostril. The patient may spit out the excess rather than swallow it. If swallowed, the composition will not harm the patient.

In other embodiments, the liquid or gelled HOCl composition may be administered in a nebulized form. As used herein, “nebulized” refers to micronized, atomized, vaporized, or aerosolized particles or a fine spray of a liquid or gel. In some embodiments, a nebulized form of the liquid or gelled HOCl composition comprises micro particles having a diameter between about 1 micron and about 1,000 microns.

Various techniques and devices for nebulizing liquids and gels are known and may be employed in practicing embodiments of the invention as to the treatment of the common cold or the treatment of an open wound or ulcer. Suitable nebulizing devices include, for example, piezoelectric nebulizers, spray bottles, metered spray pumps, metered-dose inhalers and bag-on-valve spray cans. Other devices and various techniques for nebulizing a liquid or gel will be known to one skilled in the art and are within the scope of the present invention.

While primarily described herein as useful in treating or preventing viral infection, embodiments of the present invention may also be employed in killing or destroying bacterial cells and spores. In addition, while described herein as used to treat or prevent viral infection in an individual by administering the HOCl composition to such an individual, embodiments of the invention are equally applicable to the disinfection of surfaces. For example, a liquid or gelled HOCl composition such as those described above may be applied to a cloth or wipe for disinfecting. The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A method of treating or preventing viral infection in an individual, the method comprising:
administering to the individual, via at least one nasal cavity, liquid hypochlorous acid.

2. The method of claim 1, further comprising:
administering to the individual, via the at least one nasal cavity, a quantity of gelled hypochlorous acid.

3. The method of claim 1, wherein the hypochlorous acid is produced by hydrating a powdered composition comprising:
one or more of: lithium hypochlorite, calcium hypochlorite, or sodium hypochlorite;
sodium chloride; and
an acid.

4. The method of claim 1, wherein the hypochlorous acid is produced from lithium hypochlorite, calcium hypochlorite, and/or sodium hypochlorite and ascorbic acid and/or citric acid and water resulting in a pH of 2-9.

5. The method of claim 1, wherein the hypochlorous acid is produced from lithium hypochlorite, calcium hypochlorite and/or sodium hypochlorite and ascorbic acid and/or citric acid and water resulting in a pH of 5-6.5.

6. The method of claim 1, wherein the hypochlorous acid is produced from lithium hypochlorite, calcium hypochlorite and/or sodium hypochlorite and ascorbic acid and/or citric acid, sodium chloride and water resulting in a pH of 5-6.5.

7. The method of claim 1, wherein the hypochlorous acid is produced from lithium hypochlorite, calcium hypochlorite and/or sodium hypochlorite and ascorbic acid and/or citric acid, sodium chloride and water plus a gel and/or surfactant forming inorganic emulsifier resulting in a pH of 2-9.

8. The method of claim 1, wherein the hypochlorous acid is produced from lithium hypochlorite, calcium hypochlorite and/or sodium hypochlorite and ascorbic acid and/or citric acid, sodium chloride and water plus a gel and/or surfactant forming inorganic emulsifier resulting in a pH of 5-6.5.

9. The method of claim 1, wherein the administered hypochlorous acid has hypochlorous acid content between about 10 ppm and about 500 ppm.

10. The method of claim 1, wherein the administered hypochlorous acid has hypochlorous acid content between about 50 ppm and about 150.

11. The method of claim 1, wherein the administered hypochlorous acid has sodium chloride content between about 10 ppm and about 1000 ppm.

12. The method of claim 1, wherein the administered hypochlorous acid has sodium chloride content between about 200 ppm and about 450 ppm.

13. The method of claim 1, wherein the administered hypochlorous acid is delivered by using at least one device selected from a group consisting of: a piezoelectric nebulizer, a spray bottle/can, a metered spray pump, a metered-dose inhaler, neti pot, nose rinse cleaner with powered suction and a bag-on-valve spray can.

14. The method of claim 1, wherein the viral infection includes infection by at least one virus selected from a group consisting of: a virus causing the common cold and an influenza virus.

15. The method of claim 14, wherein the virus causing the common cold is selected from a group consisting of: rhinoviruses, picornaviruses, and coronaviruses.

16. The method of claim 14, wherein the influenza virus is selected from a group consisting of: influenza virus A, influenza virus B, and influenza virus C.

17. The method of claim 14, wherein hypochlorous acid is capable of preventing the replication or colonization of the viruses that can cause the common cold, the influenza virus, or both.

18. A method of treating or preventing bacterial infection in an individual, the method comprising:
administering to the individual's wounds or ulcers a quantity of liquid hypochlorous acid.

19. The method of claim 18, further comprising: a quantity of gelled hypochlorous acid.

20. The method of claim 18, wherein the hypochlorous acid is produced from lithium hypochlorite, calcium hypochlorite and/or sodium hypochlorite and an acidic acid and water.

21. The method of claim 18, wherein the hypochlorous acid is produced from lithium hypochlorite, calcium hypochlorite and/or sodium hypochlorite and ascorbic acid and/or citric acid and water resulting in a pH of 2-9.

22. The method of claim 18, wherein the hypochlorous acid is produced from lithium hypochlorite, calcium hypochlorite and/or sodium hypochlorite and ascorbic acid and/or citric acid, and water resulting in a pH of 5-6.5.

23. The method of claim 18, wherein the hypochlorous acid is produced from lithium hypochlorite, calcium hypochlorite and/or sodium hypochlorite and ascorbic acid and/or citric acid, sodium chloride, and water resulting in a pH of 5-6.5.

24. The method of claim 18, wherein the hypochlorous acid is produced from lithium hypochlorite, calcium hypochlorite and/or sodium hypochlorite and ascorbic acid and/or citric acid, sodium chloride and water plus a gel and/or surfactant forming inorganic emulsifier.

25. The method of claim 18, wherein the hypochlorous acid is produced from lithium hypochlorite, calcium hypochlorite and/or sodium hypochlorite and ascorbic acid and/or citric acid, sodium chloride and water plus a gel forming inorganic emulsifier resulting in a pH of 2-9.

26. The method of claim 18, wherein the hypochlorous acid is produced from lithium hypochlorite, calcium hypochlorite and/or sodium hypochlorite and ascorbic acid and/or citric acid, sodium chloride and water plus a gel forming inorganic emulsifier resulting in a pH of 5-6.5.

27. The method of claim 18, wherein the administered hypochlorous acid has hypochlorous acid content between about 10 ppm and about 500 ppm.

28. The method of claim 18, wherein the administered hypochlorous acid has hypochlorous acid content between about 50 ppm and about 150 ppm.

29. The method of claim 18, wherein the administered hypochlorous acid has sodium chloride content between about 10 ppm and about 1000 ppm.

30. The method of claim 18, wherein the administered hypochlorous acid has sodium chloride content between about 200 ppm and about 450 ppm.

31. The method of claim 18, wherein the administered hypochlorous acid is delivered by using at least one device selected from a group consisting of: a piezoelectric nebulizer, a spray bottle/can, a metered spray pump, a bag-on-valve spray can or a wound dressing.

32. The method of claim 18, wherein the infection includes infection by at least one fungi/bacteria selected from a group consisting of: a fungi/bacteria that causes infection of the outer skin and the interior layers.

33. The method of claim 32, wherein the fungi/bacteria causing the infection is selected from a group consisting of: Staphylococcus aureus, Methicillin-resistant Staphylococcus aureus (MRSA), β-hemolytic streptococci, species of the genus Corynebacterium, beta-hemolytic streptococci (GABHS), Pseudomonas aeruginosa, and Dermatophyte fungi.

34. The method of claim 32, wherein hypochlorous acid is capable of preventing the replication or colonization of the fungi/bacteria that can cause the common bacterial or fungus infections in wounds or ulcers.

35. A powdered composition comprising:
one or more of: lithium hypochlorite, calcium hypochlorite, or sodium hypochlorite;
sodium chloride; and
an acid.

36. The powdered composition of claim 35 comprising lithium hypochlorite.

37. The powdered composition of claim 35 comprising calcium hypochlorite.

38. The powdered composition of claim 35 comprising sodium hypochlorite.

39. The powdered composition of claim 35 comprising both lithium hypochlorite and calcium hypochlorite.

40. The powdered composition of claim 35 comprising both lithium hypochlorite and sodium hypochlorite

41. The powdered composition of claim 35 comprising both calcium hypochlorite and sodium hypochlorite.

42. The powdered composition of claim 35 comprising lithium hypochlorite, calcium hypochlorite, and sodium hypochlorite.

43. The powdered composition of claim 35, wherein the acid includes ascorbic acid.

44. The powdered composition of claim 35, wherein the acid includes citric acid.

45. The powdered composition of claim 35, further comprising:
an inorganic emulsifier.
